(19) United States
(12) Patent Application Publication
(10) Pub. No.: US 2013/0066350 A1
(43) Pub. Date: Mar. 14, 2013

(54) TREATMENT PLANNING FOR DEEP-BRAIN NEUROMODULATION

(71) Applicant: David J. Mishelevich, Playa del Rey, CA (US)

(72) Inventor: David J. Mishelevich, Playa del Rey, CA (US)

(21) Appl. No.: 13/694,327

(22) Filed: Jan. 16, 2011

Related U.S. Application Data
(60) Provisional application No. 61/295,761, filed on Jan. 18, 2010.

(51) Int. Cl. A61B 17/32

(52) U.S. Cl. 606/169

(57) ABSTRACT

Disclosed are methods and systems for treatment planning for deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing pattern, and phase/intensity relationships mapped onto the recommended treatment-planning targets. The maps of treatment-planning targets onto which the mapping occurs can be atlas (e.g., Tailarach Atlas) based or image (e.g., fMRI or PET) based. Atlas and imaged-based maps may be representative and applied directly or scaled for the patient or may be specific to the patient.

### CROSS REFERENCE TO RELATED APPLICATIONS

This patent application claims priority to provisional patent applications Application No. 61/295,761, filed Jan. 18, 2010, entitled “TREATMENT PLANNING FOR DEEP-BRAIN NEUROMODULATION.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for treatment planning for ultrasound neuromodulation and other treatment modalities for up-regulation or down-regulation of neural activity.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996;22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels, which resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positive Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February;24(2):275-83 and Clement G T, Hynynen K (2002) A non-invasive method for focusing ultrasound through the human skull. Phys Med Biol 47: 1219-1236.) . Ultrasound can be focused to 0.5 to 2 mm as TMS to 1 cm at best.

### SUMMARY OF THE INVENTION

The invention provides methods and systems for treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Effectiveness of the application of ultrasound and other non-invasive, non-reversible modalities producing deep-brain neuromodulation such as Transcranial Magnetic Stimulation (TMS), transcranial Direct Current Stimulation (tDCS), Radio-Frequency (RF), or functional stimulation can be improved with treatment planning Treatment-plan recommendations for the application of non-reversible and/or invasive modalities such as Deep Brain Stimulation (DBS), stereotactic radiosurgery, optical stimulation, Sphenopalatine Ganglion or other localized stimulation, vagus nerve Stimulation (VNS), or future means of neuromodulation can be included.

Ultrasound transducers or other energy sources are positioned and the anticipated effects on up-regulation and/or down-regulation of their direction of energy emission, intensity, frequency, and phase/intensity relationships, dynamic-sweep configuration, and timing patterns mapped onto treatment-planning targets. The maps of treatment-planning targets onto which the mapping occurs can be atlas (e.g., Tailarach Atlas) based or image (e.g., fMRI or PET) based. Maps may be representative and applied directly or scaled for the patient or may be specific to the patient.

While rough targeting can be done with one or more of known external landmarks, or the landmarks combined with an atlas-based approach (e.g., Tailarach or other atlas used in neurosurgery) or imaging (e.g., fMRI or Positron Emission Tomography), explicit treatment planning adds benefit.

### BRIEF DESCRIPTION OF THE DRAWINGS

FIG. 1 shows a block diagram of the treatment planning

FIG. 2 illustrates a configuration of exemplar deep-brain targets.

FIG. 3 shows a diagram of a treatment plan with an ultrasound configuration mapped onto the target configuration.

FIG. 4: illustrates the treatment-planning algorithm.

### DETAILED DESCRIPTION OF THE INVENTION

Treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing and phase/intensity relationships mapped onto treatment-planning targets. The maps of treatment-planning targets onto which the mapping occurs can be atlas (e.g., Tailarach Atlas) based or image (e.g., fMRI or PET) based. Imaged-based maps may be representative and applied directly or scaled for the patient or may be specific to the patient.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes, Vagus Nerve Stimulation (VNS), and Sphenopalatine Ganglion Stimulation or other local stimulation).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency. For larger targets, larger spot sizes will be used and, depending on the shape of the targeted area, different shapes of ultrasound fields will be used.

While the description of the invention focuses on ultrasound, treatment planning can be done for therapy using other modalities (e.g., Transcranial Magnetic Stimulation (TMS), transcranial Direct Current Stimulation (tDCS), and/or Deep Brain Stimulation (DBS), Vagus Nerve Stimulation (VNS), Sphenopalatine Ganglion Stimulation and/or other local stimulation using implanted electrodes), and/or future neuromodulation means either individually or in combination.

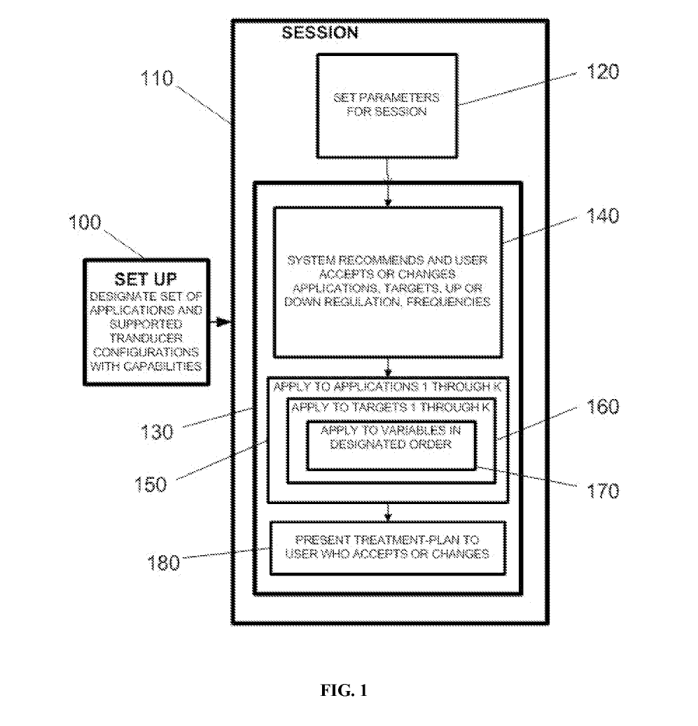

FIG. 1 shows a block diagram of the treatment planning The set-up 100 designates the set of applications to be considered as well as transducer configurations and capabilities. The session flow 110 involves setting the parameters for the session 120 that is followed by set of activities 130 in which the system recommends and the healthcare-professional user accepts or changes 140 the recommended applications, targets, up- or down-regulation, and frequencies to be used for neuromodulation. Setting of the basic parameters is followed by the application to clinical applications 1 through k 150 which incorporates application to targets 1 through k 160 within which application to variables (from among position, intensity, dynamic sweeps, and firing/timing pattern) 170 in the designated order. In step 180, the resultant treatment plan is presented to the healthcare-professional who accepts or changes the plan. Hitting multiple targets in a neural circuit in a treatment session is an important component of fostering a durable effect through Long-Term Potentiation (LTP) and/or Long-Term Depression (LTD) and is useful for acute effects as well. In addition, this approach can decrease the number of treatment sessions required for a demonstrated effect and to sustain a long-term effect. Follow-up tune-up sessions at one or more later times may be required. The treatment-planning process can be applied to other modalities or a mixture of modalities (e.g., ultrasound used simultaneously with Deep Brain Stimulation or simultaneously or sequentially with Transcranial Magnetic Stimulation). Not all variables be planned for will be same for all modalities and in some cases they may be different than those covered.

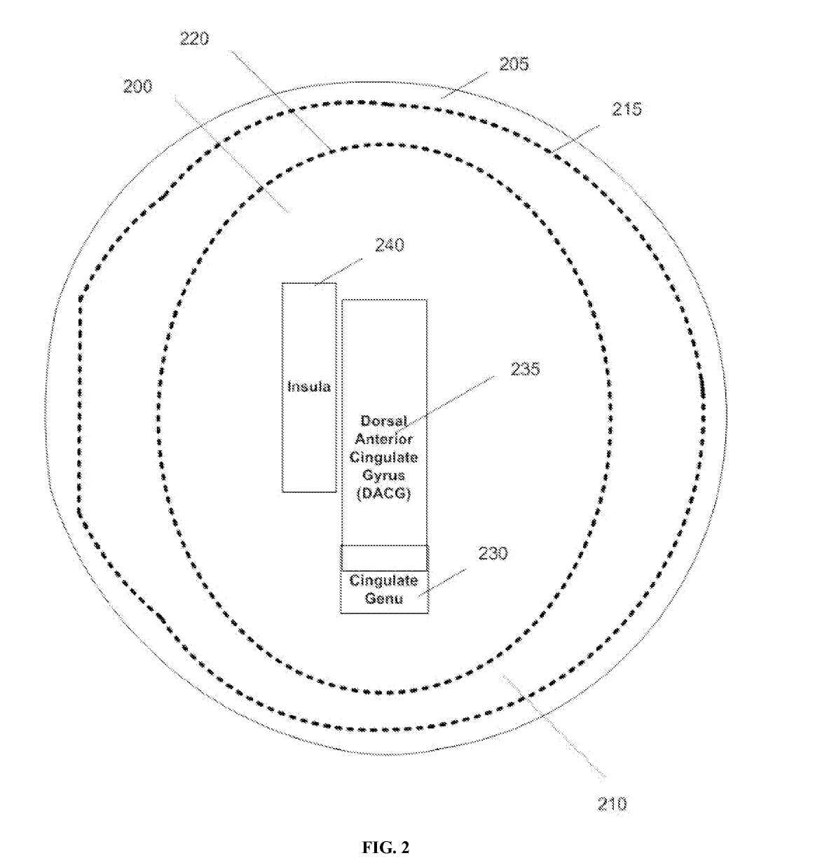

As an example of using the system, in FIG. 2, within patient head 200, three targets related to the processing of pain, the Cingulate Genu 230, Dorsal Anterior Cingulate Gyms (DACG) 235, and Insula 240. These targets, if down regulated through neuromodulation, will decrease the pain perceived by the patient. The physical context of the overall configuration is that the patient head 200 is surrounded by frame 205 on which the ultrasound transducers (not yet attached) will be fixed. Between frame 205 and patient head 200 are interposed the ultrasound-conduction medium 210 (say silicone oil housed within a containment pouch or Dermasol from California Medical Innovations) with the interface between the frame 205 and the ultrasound-conduction medium 210 filled by conduction-gel layer 215 and the interface between ultrasound-conduction medium 210 and patient head 200 filled by conduction-gel layer 220. For the ultrasound to be effectively transmitted to and through the skull and to brain targets, coupling must be put into place. This is only one configuration. In the other embodiments, the ultrasound-conduction medium and the gel layers do not have to completely surround the head, but only need be placed where the ultrasound transducers are located.

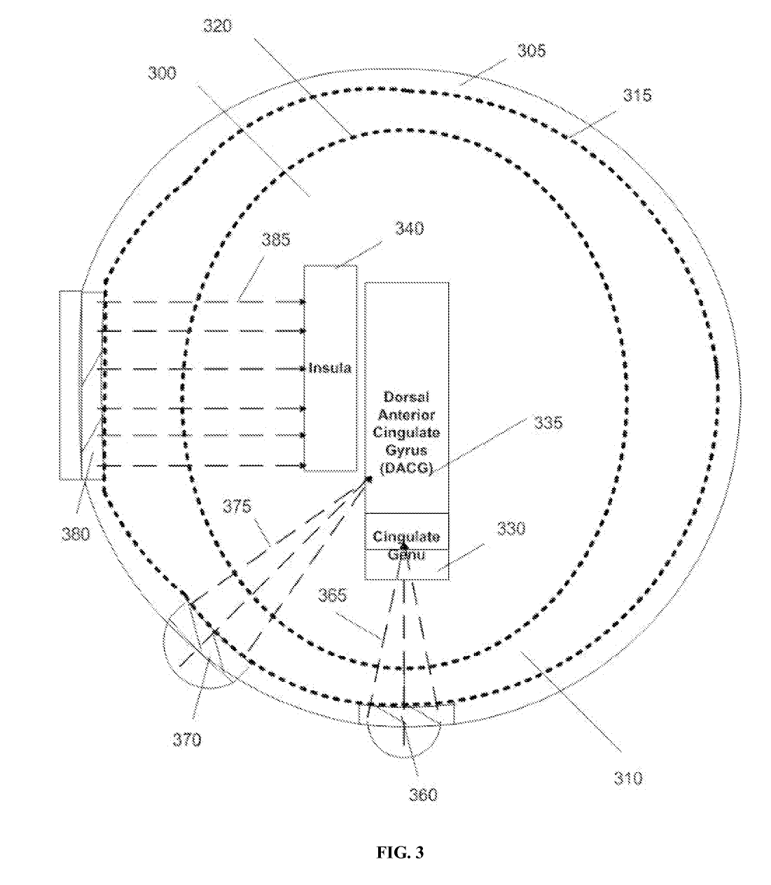

After the treatment planning of FIG. 1 is applied, the graphic as shown in FIG. 3 is displayed so the healthcare-professional can both understand the plan and place the transducers on the frame. Vertical location would be given as well (not shown) as well as saggital and coronal views displayed (not shown). In FIG. 3, patient head 300 is again surrounded by a frame 305 with interposed elements ultrasound-transmission-gel layer 320, ultrasound-transmission medium 310, and ultrasound-transmission-gel layer 315. The display shows the positioning of ultrasound transducer 360 aimed at the Cingulate Genu target 330 and the planned ultrasound field 365. In like manner, the display shows the positioning of ultrasound transducer 370 aimed at the Dorsal Anterior Cingulate Gyms (DACG) target 335 with the planned ultrasound field 375. This display also shows the positioning of ultrasound transducer 380 aimed at the Insula target 340 with the planned ultrasound field 385.

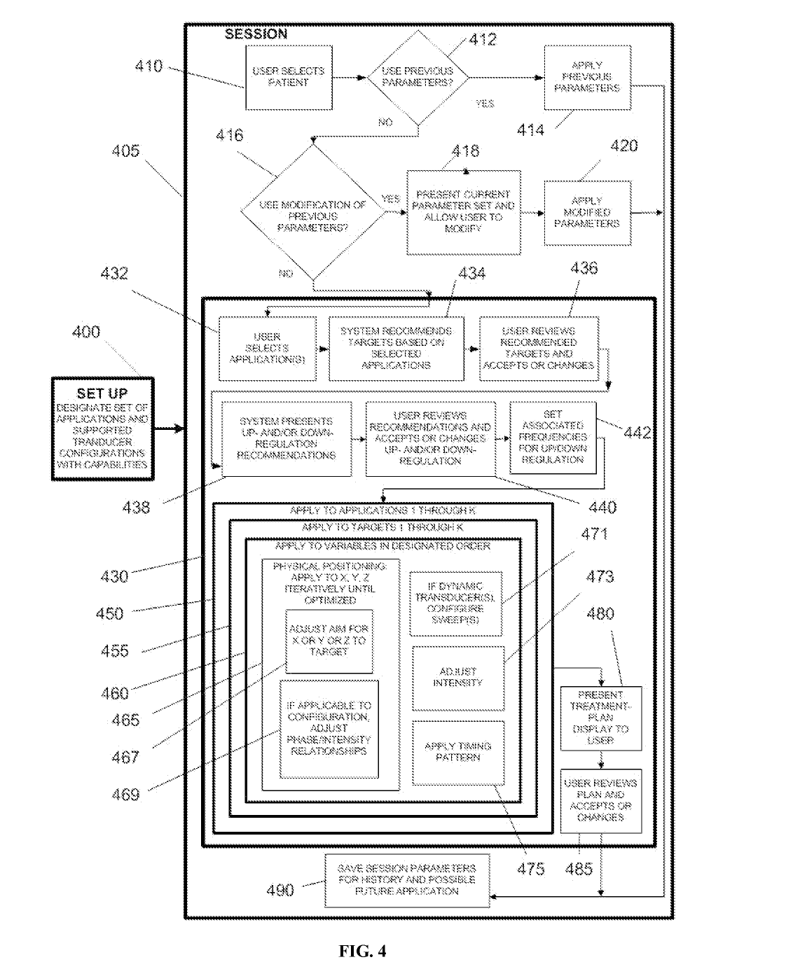

The treatment-planning process covered in FIG. 1 is shown in FIG. 4. Set up 400 includes designation of the set of applications and supported transducer configurations. Session 405 begins with step 410 where the healthcare-professional user selects the patient, which is followed by decision-step 412 as to whether or not previous parameters are to be used. If the response is yes then step 414 is executed, the application of previous parameters, after which there is step 490, saving the session parameters for the historical record and possible future application. If the response 412, use of previous parameters, is no, then decision-step 416 is executed, whether there is to be a user-supplied modification of the previous parameters. The response is yes, step 418 presents the current parameter set to the user and allows the user to modify them. Then in step 420, the modified parameters are applied, after which there is step 490, saving the session parameters for the historical record and possible future application. If the response to decision-step 416, whether there is to be a user-supplied modification of the previous parameters is no, then the flow shown in box 430 is followed. In the initial step 432 the health-professional user selects the applications to be used. This is followed by step 434, system recommending the targets based on the selected applications and step 436 where the user reviews the recommended targets and accepts or changes them. Note that for any of the healthcare-professional user's choices that are inconsistent or otherwise cannot be safely applied, the system will notify the user and offer the opportunity for corrections to be made. Step 436 is followed by step 438 in which the system presents the up- and/or down-regulation recommendations and then step 440 in which the user reviews those recommendations and accepts or changes the up- and/or down regulation designations. Down regulation means that the firing rate of the neural target has its firing rate decreased and thus is inhibited and up regulation means that the firing rate of the neural target has its firing rate increased and thus is excited. In the next step 442, the associated frequencies for up- and down-regulation are applied followed by the iterative application of the elements in box 450 in which in the outer loop the process is applied to applications 1 through k. In succeeding inner loop 455, the process is applied iteratively to targets 1 through k and in its succeeding inner loop 460; the process is applied iteratively to variables in the designated order. In step 465, the physical positioning is applied to x, y, and z iteratively until optimized with 467 adjustment of the aim to target, and 469, if applicable to the configuration, adjustment of the phase/intensity relationships for beam steering and/or focus. Step 471, configuring of sweep(s) is executed if there are dynamic transducers. In step 473, the intensity is adjusted, and the firing/timing pattern applied in 475. The ultrasonic firing/timing patterns can be tailored to the response type of a target or the various targets hit within a given neural circuit. In the output of box 450, in step 480, the treatment-plan display is presented to the user followed by step 485 in which the user reviews the plan and accepts or changes it. Again, if the plan is inconsistent or cannot otherwise be safely executed, the system will notify the user and offer the opportunity for corrections to be made. Following acceptance of the treatment plan, there is step 490, saving the session parameters for the historical record and possible future application.

The invention can be applied to individual, simultaneous, or sequential neuromodulation of one or a plurality of targets including, but not limited to NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

The invention can be applied to a one or a plurality of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to one or a plurality of cognitive enhancement, hedonic stimulation, enhancement of neural plasticity, improvement in wakefulness, brain mapping, diagnostic applications, and research functions. In addition to stimulation or depression of individual targets, the invention can be used to globally depress neural activity, which can have benefits, for example, in the early treatment of head trauma or other insults to the brain.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method for treatment planning for neuromodulation of deep-brain targets using ultrasound neuromodulation, the method comprising:
setting up sets of applications and supported transducer configurations with associated capabilities,
executing treatment-planning sessions including
setting parameters for the session,
system recommendations and user acceptance of changes to applications, targets, up- or down-regulation, stimulation frequencies,
iterating through set of applications;
iterating through set of targets;
iterating through and applying in designated order one or more variables
selected from the group consisting of position, intensity, firing-timing pattern, phase/intensity relationships, dynamic sweeps;
presenting treatment plan to user who accepts or changes;
whereby the treatment to be delivered is tailored to the patient.

2. The method of claim 1 where the one or plurality of treatment modalities are selected from the group consisting of ultrasound, Deep Brain Stimulation, stereotactic radiosurgery, optical stimulation, Sphenopalatine Ganglion stimulation, other localized stimulation, vagus nerve stimulation, and future means of neuromodulation.

3. The method of claim 1 where the maps of treatment-planning targets onto which the mapping are selected from the group consisting of atlas based or image based.

4. The method of claim 3 where the maps are selected from the group consisting of specific to the patient, representative and applied directly, and representative where scaled for the patient.

5. The method of claim 1, wherein the one or a plurality of target brain regions involved in the treatment plan are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, and any of the tracts between the brain targets.

6. The method of claim 1, wherein the one or plurality of disorders for which treatment is planned are selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

7. The method of claim 1 wherein the one or a plurality of application for which treatment is planned are selected from the group consisting of: cognitive enhancement, hedonic stimulation, enhancement of neural plasticity, improvement in wakefulness, brain mapping, diagnostic applications, and research functions.

8. A system for treatment planning for neuromodulation of deep-brain targets using ultrasound neuromodulation, the method comprising:
setting up sets of applications and supported transducer configurations with associated capabilities,
executing treatment-planning sessions including
setting parameters for the session,
system recommendations and user acceptance of changes to applications,
targets, up- or down-regulation, stimulation frequencies,
iterating through set of applications;
iterating through set of targets;
iterating through and applying in designated order one or more variables selected from the group consisting of position, intensity, firing-timing pattern, phase/intensity relationships, dynamic sweeps;
presenting treatment plan to user who accepts or changes;
whereby the treatment to be delivered is tailored to the patient.

9. The system of claim 8 where the one or plurality of treatment modalities are selected from the group consisting of ultrasound, Deep Brain Stimulation, stereotactic radiosurgery, optical stimulation, Sphenopalatine Ganglion stimulation, other localized stimulation, vagus nerve stimulation, and future means of neuromodulation.

10. The system of claim 8 where the maps of treatment-planning targets onto which the mapping are selected from the group consisting of atlas based or image based.

11. The system of claim 9 where the maps are selected from the group consisting of specific to the patient, representative and applied directly, and representative where scaled for the patient.

12. The system of claim 8, wherein the one or a plurality of target brain regions involved in the treatment plan are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, and any of the tracts between the brain targets.

13. The system of claim 8, wherein the one or plurality of disorders for which treatment is planned are selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

14. The system of claim 8 wherein the one or a plurality of application for which treatment is planned are selected from the group consisting of: cognitive enhancement, hedonic stimulation, enhancement of neural plasticity, improvement in wakefulness, brain mapping, diagnostic applications, and research functions.
